(19) United States
(12) Patent Application Publication
(10) Pub. No.: US 2021/0369902 A1
(43) Pub. Date: Dec. 2, 2021

(54) COSMETICS CONTAINER, INNER LID, TUBULAR MEMBER, ROD-SHAPED MEMBER, AND MAIN BODY SIDE INNER LID

(71) Applicant: AMG Co., Ltd., Tokyo (JP)

(72) Inventor: Hiroki HASEGAWA, Tokyo (JP)

(21) Appl. No.: 17/271,406

(22) PCT Filed: Mar. 30, 2020
(86) PCT No.: PCT/JP2020/014694

§ 371 Date: Feb. 25, 2021

(51) Int. Cl. A61L 2/238; A45D 33/24; A45D 40/00; A61L 2/232

(52) U.S. Cl. CPC A61L 2/238; A45D 33/24; A61L 2202/23; A61L 2/232; A45D 2200/05; A45D 40/0068

(57) ABSTRACT

[Problem to be Solved]

To provide a cosmetics container or the like capable of reducing bacteria in cosmetic product.

[Solution]

A container main body 20 of the cosmetics container of the present invention is configured of a main body portion 22 that configures the basic structure of the container main body 20 and an inner surface portion 24 that configures the inner surface of the container main body 20. The inner surface portion 24 is configured as a sterilizing portion in which metal fine particles having an effect of reducing bacteria are dispersed in a resin and bacteria are reduced by the action of the fine particles. The fine particles are configured so as not to be exposed on the surface of the inner surface portion 24. The type of metal configuring the sterilizing portion is defined according to the type of bacteria that is assumed to become a problem at each position on the inner surface portion 24.

### TECHNICAL FIELD

The present invention is related to a cosmetics container, an inner lid, a tubular member, a rod-shaped member, and a main body side inner lid.

### BACKGROUND ART

It is important for a cosmetics container to be able to preserve and maintain the quality of a cosmetic product such as foundation.

Therefore, preservatives (paraben, etc.) and additives such as phenoxyethanol having an antiseptic effect may be added to a cosmetic product. However, it is preferable not to use preservatives or additives. Meanwhile, a technique for filling cosmetic product in a container having a small capacity sufficient for filling a single use amount has been proposed (for example, Patent Literature 1).

### CITATION LIST

### Patent Literature

[Patent Literature 1] JP 4627562

### SUMMARY OF INVENTION

### Technical Problem

When the amount used at one time is extremely small, filling cosmetic product in a container having a small capacity sufficient for filling a one-time use amount of cosmetic product requires a large number of containers. In addition, the amount used at one time may be different for different people, and even for the same person, it may be different depending on the situation, so that the amount of cosmetic product in the container may be excessive or insufficient, and eventually cosmetic product may be wasted.

Based on the above, the present invention provides a cosmetics container, an inner lid, a tubular member, a rod-shaped member, and a main body side inner lid, which can reduce bacteria in a cosmetic product.

### Solution to Problem

A first invention is a cosmetics container, which is a cosmetics container having a container main body for holding a cosmetic product, and a lid body which detachably engages with the container main body; wherein the container main body is configured of a main body portion configuring the basic structure of the container main body and an inner surface portion configuring an inner surface of the container main body; the inner surface portion is configured as a sterilizing portion in which metal fine particles having an effect of reducing bacteria are dispersed in a resin and bacteria are reduced by the action of the fine particles; the fine particles are configured so as not to be exposed on the surface of the inner surface portion; and the type of the metal configuring the sterilizing portion is defined in accordance with the type of bacteria assumed to become a problem at each position on the inner surface portion.

According to the configuration of the first invention, the inner surface portion is formed by dispersing metal fine particles having an effect of reducing bacteria in a resin, and since the surface of the fine particles is not exposed on the surface of the inner surface portion, metal does not come into direct contact with the cosmetic product. Therefore, there is no issue with the metal causing a chemical reaction with the components of the cosmetic product or denaturing the cosmetic product. As described in detail in the present specification, it has been confirmed through testing that it is possible to reduce bacteria even when the metal does not come into direct contact with the cosmetic product. Furthermore, the inventor of the present invention has found that the types of bacteria which can be effectively reduced differ depending on the type of metal. The inventor of the present invention has found, for example, that there are metals which can effectively reduce aerobic bacteria and metals which can effectively reduce anaerobic bacteria. In addition, the inventor of the present invention determined the type of metal effective for reducing the type of bacteria assumed to become a problem at a position on the inner surface portion and came up with the technology for dispersing the determined metal fine particles in the sterilizing portion (hereinafter referred to as “selective dispersion”). In this regard, according to the configuration of the first invention, since the types of metals configuring the sterilizing portion are defined according to the types of bacteria that are assumed to become problematic at each position on the inner surface portion, it is possible to realize selective dispersion.

A second invention is the cosmetics container according to the configuration of the first invention, wherein the inner surface portion includes the metal fine particles of a plurality of types; and at each position on the inner surface portion, the content of fine particles of each metal is defined relative to the total content of the metal fine particles in accordance with the type of bacteria assumed to become a problem.

The type of bacteria assumed to become a problem at a specific position on the inner surface is not limited to only one type. The inventor of the present invention has found that there is a plurality of types of bacteria that may be assumed to become a problem at a specific position on the inner surface, and that the types of bacteria that can be relatively abundant differ depending on the specific position. For example, aerobic bacteria and anaerobic bacteria may be problematic at any position on the inner surface portion, but it is assumed that higher up there are more aerobic bacteria than anaerobic bacteria and lower down there are more anaerobic bacteria than aerobic bacteria. In addition, the inventor of the present invention came up with the technology for blending a plurality of types of metal fine particles for effectively reducing each type of bacteria according to the plurality of types of bacteria that are assumed to become a problem at a specific position and dispersion in the sterilizing portion (hereinafter referred to as “load distribution”). In this regard, according to the configuration of the second invention, since the inner surface portion contains fine particles of a plurality of types of metals, and the content of the fine particles of each metal is defined according to the type of bacteria assumed to become problematic at each position of the inner surface portion, it is possible to realize load distribution. Note that load distribution is subordinate concept technology of selective distribution.

A third invention is the cosmetics container according to the configuration of the second invention, wherein each position on the inner surface portion includes an upper portion located relatively upper and a lower portion located relatively lower; the type of bacteria expected to be relatively more likely to become a problem in the upper portion is aerobic bacteria, and the type of bacteria expected to be relatively more likely to become a problem in the lower portion is anaerobic bacteria; the upper portion is configured so that the content of the metal fine particles having a property of effectively reducing aerobic bacteria is increased; and the lower portion is configured so that the content of the metal fine particles having a property of effectively reducing anaerobic bacteria is increased.

According to the configuration of the third invention, at each position on the inner surface portion, the type of metal is determined according to the type of bacteria assumed to be relatively more likely to become a problem, and by increasing that metal fine particle content, it is possible to effectively reduce the bacteria assumed to be relatively more likely to become a problem at each position.

A fourth invention is the cosmetics container according to the configuration of the second invention, wherein the inner surface portion is configured so that, from the upper side toward the lower side, the content of the metal fine particles having a property of effectively reducing aerobic bacteria decreases and the content of the metal fine particles having a property of effectively reducing anaerobic bacteria increases.

In a state where the cosmetic product is being held in the container main body, aerobic bacteria are more likely to be a problem in the upper portion of the container main body, and anaerobic bacteria are more likely to be a problem in the lower portion of the container main body. In this regard, according to the configuration of the fourth invention, since the content of metal fine particles capable of effectively reducing bacteria is defined according to the possibility that aerobic bacteria and anaerobic bacteria may become a problem, it is possible to effectively reduce bacteria at each position.

A fifth invention is the cosmetics container according to the configuration of any one of the first through fourth inventions, wherein in the inner surface portion, the metal fine particles are configured to increase in content as they approach a position close to a surface where the cosmetic product comes into contact with the inner surface portion.

The closer the metal fine particles are distributed to a surface in contact with the cosmetic product, the greater the effect of reducing bacteria. In this regard, according to the configuration of the fifth invention, since the metal fine particles are configured so that the content of the metal fine particles increases closer to the surface of the inner surface portion in contact with the cosmetic product, it is possible to effectively reduce bacteria.

A sixth invention is the cosmetics container according to the configuration of any one of the first through fifth inventions, wherein a thickness of the inner surface portion is formed to be a thickness within a predetermined range defined in relation to the size of the outer shape of the fine particles.

According to the configuration of the sixth invention, for example, focusing on the specific surface area of the fine particles, it is possible to utilize more effectively the bacterial reduction effect of the fine particle, for example, by making the inner surface portion thinner as the smaller the diameter of the fine particles, and by defining the thickness of the inner surface portion based on the total surface area of the fine particle group configured from many fine particles.

A seventh invention is the cosmetics container according to the configuration of any one of the first through sixth inventions, wherein the inner surface portion has a side surface portion and a bottom surface portion with the side surface portion and/or the bottom surface portion formed as an uneven surface having a plurality of convex portions and concave portions.

According to the configuration of the seventh invention, compared to the case where the inner surface portion is a flat surface or a curved surface without unevenness, since it is possible to configure larger the area where the inner surface portion comes into contact with the cosmetic product, it is possible to utilize even more effectively the bacteria reducing effect of the fine particles.

An eighth invention is the cosmetics container according to the configuration of any one of the first through the seventh inventions, wherein the inner surface portion has a side surface portion and a bottom surface portion, and only one of the side surface portion or the bottom surface portion is configured as a sterilizing portion.

According to the configuration of the eighth invention, it is possible to effectively reduce bacteria by, for example, in the case of a flat container main body, configuring only the bottom surface portion as a sterilizing portion, and in the case of an elongated container main body, configuring only the side surface portion as a sterilizing portion.

A ninth invention is a cosmetics container, which is a cosmetics container having a container main body for holding a cosmetic product and a lid body which detachably engages with the container main body; wherein an inner lid is connected to the lid body via a connecting member; the inner lid is configured from an inner lid main body configuring an upper portion, and an inner lid lower portion configuring a lower portion; the inner lid lower portion is configured as a sterilizing portion in which metal fine particles having an effect of reducing bacteria are dispersed in a resin and bacteria are reduced by the action of the fine particles; the inner lid lower portion is configured so that the fine particles are not exposed on the surface of the inner lid lower portion; a length of the connecting member is defined as an adjustable length which allows the inner lid to come into contact with a bottom surface of the container main body; by adjusting the length of the connecting member, the inner lid lower portion is configured to come into contact with the cosmetic product regardless of the amount of the cosmetic product held in the container main body; and the inner lid lower portion is configured to have, among the plurality of types of the metal fine particles, a large content of the metal fine particles having a property of being able to effectively reduce aerobic bacteria.

Bacteria may grow on the upper surface of the cosmetic product inside the container main body that comes into contact with the external air or the user's hands. In this regard, according to the configuration of the ninth invention, when the lid body is engaged with the container main body, since the lower part of the inner lid configured as the sterilizing part comes into contact with the upper surface of the cosmetic product, it is possible to effectively reduce the bacteria which occurs on the upper surface of the cosmetic product.

A tenth invention is a cosmetics container, which is a cosmetics container having a container main body for holding cosmetic product and a lid body which detachably engages with the container main body; wherein, to the lid body, a tubular member is connected; a surface layer portion of the tubular member is configured as a sterilizing portion in which metal fine particles having an effect of reducing bacteria are dispersed in a resin and bacteria are reduced by the action of the fine particles; a surface layer portion of the tubular member is configured so that the fine particles are not exposed on the surface of the surface layer portion; a length of the tubular member is defined as a length allowing contact with a bottom surface of the container main body; regardless of the amount of the cosmetic product held in the container main body, the surface layer portion of an inner surface and an outer surface of the tubular member are configured to be in contact with the cosmetic product; and the type of the metal configuring the sterilizing portion is defined according to the type of bacteria assumed to become a problem at each position of the surface layer portion.

According to the configuration of the tenth invention, when the lid body engages with the container main body, since the surface layer portion of the inner surface and the outer surface of the tubular member configured as the sterilizing portion comes into contact with the cosmetic product, it is possible to effectively reduce bacteria which develops in the cosmetic product.

An eleventh invention is a cosmetics container, which is a cosmetics container having a container main body for holding a cosmetic product and a lid body which detachably engages with the container main body; wherein, to the lid body, a least one rod-shaped member is connected; a surface layer portion of the rod-shaped member is configured as a sterilizing portion in which metal fine particles having an effect of reducing bacteria are dispersed in a resin and bacteria are reduced by the action of the fine particles; the surface layer portion is configured so that the fine particles are not exposed on the surface of the surface layer portion; the rod-shaped member is connected at a position deviating from the center of the lid body, and as the lid body is rotated for attachment/detachment to/from the container main body, is configured to rotate and stir the cosmetic product held in the container main body; and the type of the metal configuring the sterilizing portion is defined according to the type of bacteria assumed to become a problem at each position of the surface layer portion.

According to the configuration of the eleventh invention, when the lid body engages with the container main body, since the surface layer portion of the rod-shaped member configured as the sterilizing portion comes into contact with the cosmetic product, it is possible to reduce more effectively the bacteria which develops in the cosmetic product. Further, as the lid body is attached to or detached from the container main body, the cosmetic product is stirred by the rod-shaped member so that the rod-shaped member comes into contact with many portions of the cosmetic product, and it is possible to reduce bacteria more effectively.

A twelfth invention is a cosmetics container, which is a cosmetics container having a container main body for holding a cosmetic product and a lid body which detachably engages with the container main body; wherein on the container main body, a main body side inner lid is arranged; the main body side inner lid has a main body side inner lid main body portion configuring an upper portion and a main body side inner lid lower portion configuring a lower portion, and is formed with a through hole passing through in the vertical direction the main body side inner lid; the main body side inner lid lower portion is configured as a sterilizing portion in which metal fine particles having an effect of reducing bacteria are dispersed in a resin and bacteria are reduced by the action of the fine particles; the main body side inner lid lower portion is configured so that the fine particles are not exposed on the surface of the main body side inner lid lower portion; the main body side inner lid lower portion is configured to come into contact with the cosmetic product, and by moving the main body side inner lid so as to push on the cosmetic product, the cosmetic product is able to be pushed out from the through hole; and the main body side inner lid lower portion is configured so that of the plurality of types of the metal fine particle, the content of the metal fine particles having a property of effectively reducing aerobic bacteria is large.

According to the configuration of the twelfth invention, the sterilizing portion of the container main body and the main body side inner lid lower portion reduces bacteria in the cosmetic product, and moreover, since only the cosmetic product used by the user is exposed to the outside, the possibility of being contaminated with bacteria is low.

A thirteenth invention is an inner lid, which is an inner lid connected to a lid body which detachably engages with a container main body for holding a cosmetic product; wherein the inner lid is connected to the lid body via a connecting member, and configured of an inner lid main body portion configuring an upper portion and an inner lid lower portion configuring the lower portion; the inner lid lower portion is configured as a sterilizing portion in which metal fine particles having an effect of reducing bacteria are dispersed in the resin and bacteria are reduced by the action of the fine particles; the inner lid lower portion is configured so that the fine particles are not exposed on the surface of the inner lid lower portion; the length of the connecting member is defined as an adjustable length that allows the inner lid to come into contact with a bottom surface of the container main body; by adjusting the length of the connecting member, the inner lid lower portion is configured to come into contact with the cosmetic product regardless of the amount of the cosmetic product held in the container main body; and the inner lid lower portion is configured so that of the plurality of types of the metal fine particles, the content of the metal fine particles having a property of effectively reducing aerobic bacteria is large.

A fourteenth invention is a tubular member, which is a tubular member connected to a lid body that detachably engages with a container main body for holding cosmetic product; wherein a surface layer portion of the tubular member is configured as a sterilizing portion in which metal fine particles having an effect of reducing bacteria are dispersed in a resin and bacteria are reduced by the action of the fine particles; the surface layer portion is configured so that the fine particles are not exposed on the surface of the surface layer portion; a length of the tubular member is defined as a length that allows contact with a bottom surface of the container main body; the surface layer portions of an inner surface and an outer surface of the tubular member are configured to be in contact with the cosmetic product regardless of the amount of the cosmetic product held in the container main body; and the metal configuring the sterilizing portion is defined according to the type of bacteria assumed to become a problem at each position on the surface layer portion.

A fifteenth invention is a rod-shaped member, which is a rod-shaped member connected to a lid body that detachably engages with a container main body for holding cosmetic product; wherein a surface layer portion of the rod-shaped member is configured as a sterilizing portion in which metal fine particles having an effect of reducing bacteria are dispersed in a resin and bacteria are reduced by the action of the fine particles; the surface layer portion is configured so that the fine particles are not exposed on the surface of the surface layer portion; the rod-shaped member is connected at a position deviating from the center of the lid body, and as the lid body is rotated for attachment/detachment to/from the container main body, is configured to rotate and stir the cosmetic product held in the container main body; and the metal configuring the sterilizing portion is defined according to the type of bacteria assumed to become a problem at each position on the surface layer portion.

A sixteenth invention is a main body side inner lid, which is a main body side inner lid that is arranged on a container main body for holding cosmetic product; wherein the main body side inner lid is configured by integrally forming a main body side inner lid main body portion configuring an upper portion and a main body side inner lid lower portion configuring a lower portion, and a through hole is formed so as to pass through in the vertical direction the main body side inner lid; the main body side inner lid lower portion is configured as a sterilizing portion in which metal fine particles having an effect of reducing bacteria are dispersed in a resin and bacteria are reduced by the action of the fine particles; the main body side inner lid lower portion is configured so that the fine particles are not exposed on the surface of the main body side inner lid lower portion; the main body side inner lid lower portion is configured to come into contact with the cosmetic product, and by moving the main body side inner lid so as to push on the cosmetic product, the cosmetic product is able to be pushed out from the through hole; and the inner lid lower portion is configured so that of the plurality of types of the metal fine particles, the content of the metal fine particles having a property of effectively reducing aerobic bacteria is large.

### Effect of the Invention

According to the cosmetics container according to the present invention, it is possible to reduce bacteria in a cosmetic product.

### BRIEF DESCRIPTION OF THE DRAWINGS

FIG. 1 is a schematic perspective view of a cosmetics container according to a first embodiment of the present invention.

FIG. 2 is a schematic perspective view of a container main body.

FIG. 3 is a schematic perspective view of a lid body.

FIG. 4 is a schematic exploded view of a container main body.

FIG. 5 is a schematic side view of a container main body.

FIG. 6 is a schematic plan view of a container main body.

FIG. 7 is a schematic cross-sectional view of a container main body.

FIG. 8 is a schematic cross-sectional view of an inner surface portion.

FIG. 9 is an enlarged conceptual view illustrating a cross section of an upper portion of an inner surface portion.

FIG. 10 is an enlarged conceptual view illustrating a cross section of a lower portion of an inner surface portion.

FIG. 11 is a schematic cross-sectional view illustrating the state in which cosmetic product is stored in a container main body.

FIG. 12 is a conceptual view illustrating the action of an inner surface portion in state in which cosmetic product is stored in a container main body.

FIG. 13 is an enlarged conceptual view illustrating the action of an upper portion of an inner surface portion.

FIG. 14 is an enlarged conceptual view illustrating the action of a lower portion of an inner surface portion.

FIG. 15 is a chart illustrating testing results.

FIG. 16 is a schematic cross-sectional view of an inner surface portion according to a second embodiment of the present invention.

FIG. 17 is an enlarged conceptual view illustrating a cross section of an upper portion of an inner surface portion.

FIG. 18 is an enlarged conceptual view illustrating a cross section of a lower portion of an inner surface portion.

FIG. 19 is a schematic cross-sectional view of an inner surface portion according to a third embodiment of the present invention.

FIG. 20 is an enlarged conceptual view illustrating a cross section of an inner surface portion.

FIG. 21 is a graph which conceptually illustrates the content of copper fine particles relative to the content of all metal fine particles at a position on an inner surface portion in the vertical direction.

FIG. 22 is a schematic cross-sectional view of an inner surface portion according to a fourth embodiment of the present invention.

FIG. 23 is an enlarged conceptual view illustrating a cross section of an inner surface portion.

FIG. 24 is a schematic plan view of a container main body according to a fifth embodiment of the present invention.

FIG. 25 is a schematic cross-sectional view of a cosmetics container according to a sixth embodiment of the present invention.

FIG. 26 is a schematic cross-sectional view of a cosmetics container according to a seventh embodiment of the present invention.

FIG. 27 is a schematic perspective view illustrating a lid body of a cosmetics container according to an eighth embodiment of the present invention.

FIG. 28 is a schematic view illustrating the operation of an inner lid.

FIG. 29 is a schematic view illustrating the operation of an inner lid.

FIG. 30 is a schematic perspective view illustrating a lid body of a cosmetics container according to a ninth embodiment of the present invention.

FIG. 31 is a schematic view illustrating the layer structure of a peripheral wall of an upper portion of a tubular member.

FIG. 32 is a schematic view illustrating the layer structure of a peripheral wall of a lower portion of a tubular member.

FIG. 33 is a schematic view illustrating the operation of a tubular member.

FIG. 34 is a schematic view illustrating the operation of a tubular member.

FIG. 35 is a schematic perspective view illustrating a lid body of a cosmetics container according to a tenth embodiment of the present invention.

FIG. 36 is a schematic view illustrating the layer structure of a rod-shaped member.

FIG. 37 is a schematic bottom view illustrating the arrangement state of a rod-shaped member.

FIG. 38 is a schematic view illustrating the action of a rod-shaped member.

FIG. 39 is a schematic cross-sectional view of a container main body according to an eleventh embodiment of the present invention.

FIG. 40 is a schematic perspective view of an inner lid.

FIG. 41 is a schematic bottom view of an inner lid.

FIG. 42 is a schematic cross-sectional view illustrating the use state of a container main body.

FIG. 43 is a schematic cross-sectional view illustrating a first reference example.

FIG. 44 is a schematic cross-sectional view illustrating a second reference example.

### DESCRIPTION OF EMBODIMENTS

Preferred embodiments of the present invention will be described below with reference to the drawings. Note that description of configurations that can be appropriately implemented by those skilled in the art will be omitted, and only the basic configuration of the present invention will be described.

### First Embodiment

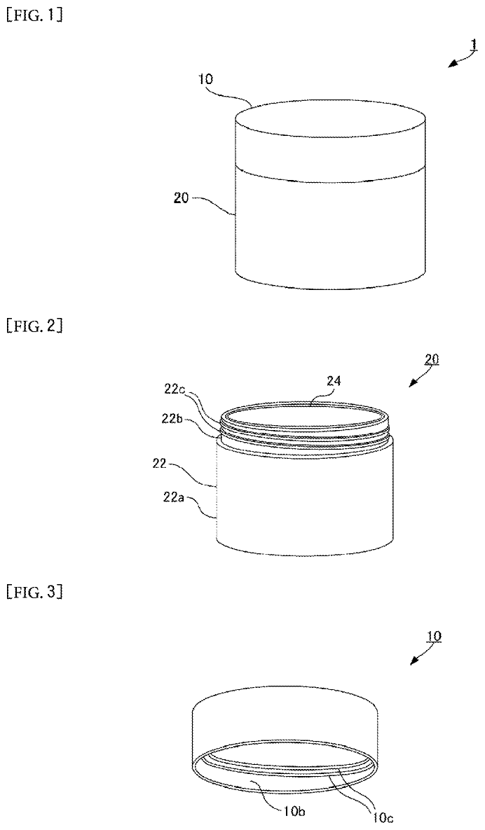

As illustrated in FIG. 1, a cosmetics container 1 has a lid body 10 and a container main body 20. The container main body 20 has a hollow structure, and a cosmetic product is stored in the space of the hollow structure. The lid body 10 also has a hollow structure. In the present specification, the direction connecting the lid body 10 and the container main body 20 is referred to as “vertical direction”, the direction of the lid body 10 is referred to as “upper side”, and the direction of the container main body 20 is referred to as “lower side”.

As illustrated in FIGS. 2, 4, 6 and 7, the container main body 20 is configured of a main body portion 22 configuring the basic structure of the container main body 20 and an inner surface portion 24. The main body portion 22 configures the outer layer portion of the container main body 20, and the inner surface portion 24 configures the inner layer portion of the container main body. The inner surface portion 24 is formed inside the main body portion 22 in a state of being in contact with the main body portion 22. The main body portion 22 is formed, for example, by injection molding of a resin. As for the resin configuring the main body portion 22, for example, a polyolefin-based resin such as polypropylene, a polystyrene-based resin, or a polyester-based resin can be used.

The inner surface portion 24 is formed of a resin. As for the type of resin configuring the inner surface portion 24, the same resin as that of the main body portion 22 may be adopted, or a different resin may be adopted. A large number of metal fine particles having an effect of reducing bacteria are dispersed and present in the resin configuring the inner surface portion 24. The inner surface portion 24 is configured as a sterilizing portion. The inner surface portion 24 is configured so that the metal fine particles are not exposed on the surface. The inner surface portion 24 is formed, for example, by mixing resin powder and a large number of metal fine particles, adding an appropriate coupling material such as a silane coupling material, and other additives as necessary, and injection molding. In the present specification, the meaning of “reducing bacteria” includes the meaning of preventing the growth of bacteria as well as reducing or eliminating bacteria that have developed.

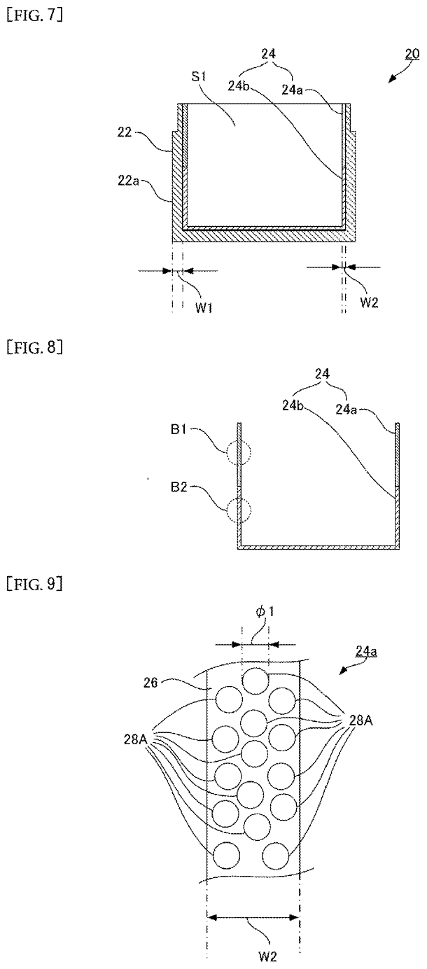

As illustrated in FIGS. 4 and 7, the inner surface portion 24 is configured of an upper portion 24a located relatively upper and a lower portion 24b located relatively lower.

The main body portion 22 and the inner surface portion 24 are integrally formed. While any appropriate manufacturing method can be used for integral formation, for example, a molding method such as insert molding, in-mold molding, or two-color molding may be used.

The method of forming the inner surface portion 24 is not limited to the above method. For example, unlike the present embodiment, the inner surface portion 24 may be connected to the inside of the main body portion 22 with a sheet (foil) in which the above-mentioned metal is dispersed in a resin. Further, the inner surface portion 24 may be coated on the inside of the main body portion 22 by forming a coating agent from the resin and the above-mentioned metal.

Metals having the effect of reducing bacteria are, for example, copper (Cu) and silver (Ag). Metal having the effect of reducing bacteria is not limited to copper and silver, and may be zinc, for example. The upper portion 24a of the inner surface portion 24 is formed by dispersing copper fine particles in the resin. The lower portion 24b is formed by dispersing silver fine particles in the resin.

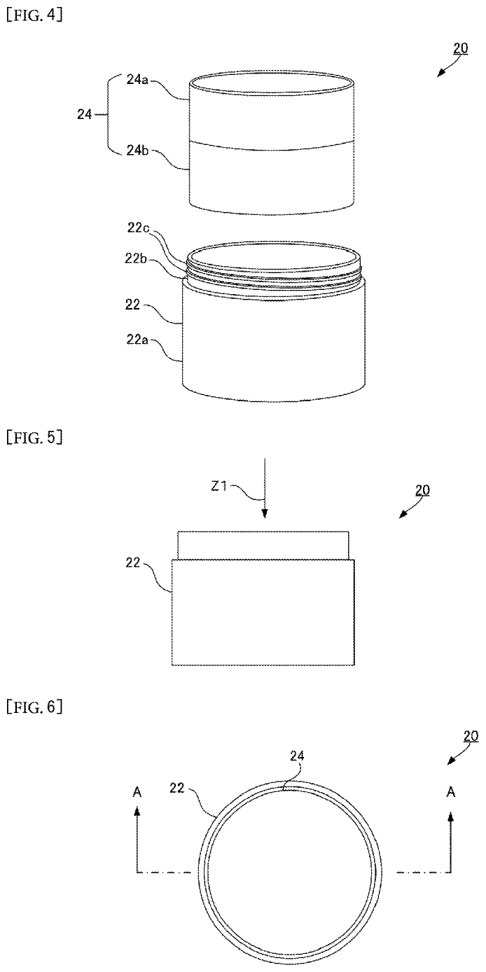

As illustrated in FIGS. 2 and 4, the main body portion 22 is configured of a base portion 22a and a connecting portion 22b. A male threaded portion 22c is formed on the connecting portion 22b. As illustrated in FIG. 3, a female threaded portion 10c is formed on the inner surface 10b of the lid body 10. The male threaded portion 22c of the connecting portion 22b engages with the female threaded portion 10c formed on the inner surface of the lid body 10, so that the lid body 10 and the container main body 20 are detachably engaged with each other.

FIG. 6 is a schematic plan view of the container main body 20 of FIG. 5 as viewed from the direction of arrow Z1. FIG. 7 is a schematic cross-sectional view taken along a line AA of the container main body 20 illustrated in FIG. 6. As illustrated in FIG. 7, the thickness W1 of the base portion 22a of the main body portion 22 is larger than the thickness W2 of the inner surface portion 24. In other words, the thickness W2 of the inner surface portion 24 is smaller than the thickness W1 of the main body portion 22.

The ratio C (W2/W1) of the thickness W2 of the inner surface portion 24 to the thickness W1 of the main body portion 22 is defined to be 0.001 or more and 0.050 or less, and preferably 0.002 or more and 0.008 or less.

For example, the thickness W1 of the main body portion 22 is 3 millimeters (mm), the thickness W2 of the inner surface portion 24 is 20 micrometers (μm), and the ratio C (W2/W1) is 0.007. As described above, the inner surface portion 24 is configured as a sterilizing portion for reducing bacteria. Meanwhile, the main body portion 22 is configured as a non-sterile portion that does not have the effect of reducing bacteria.

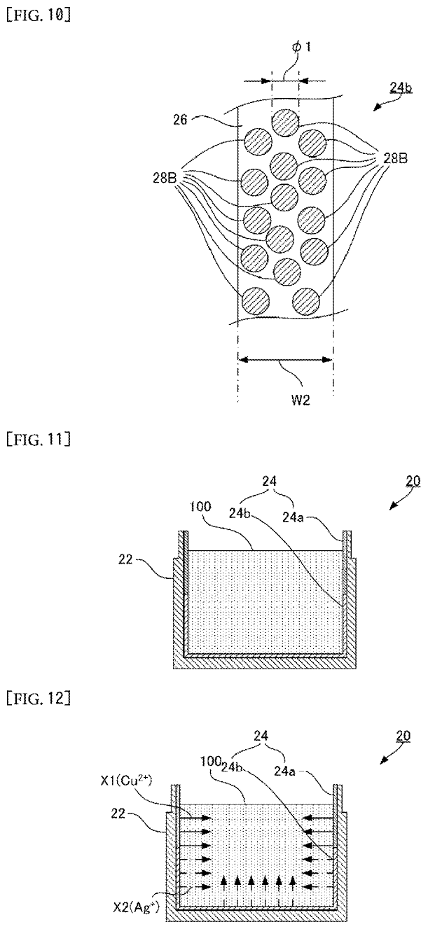

FIG. 9 is a conceptual view illustrating in an enlarged manner a portion B1 of the upper portion 24a of the inner surface portion 24 illustrated in FIG. 8. The upper portion 24a is configured by dispersing copper fine particles 28A in the resin 26. The fine particles 28A are not exposed on the surface of the upper portion 24a and are covered with the resin 26. In the present specification, it is given that “copper” includes copper and copper oxide.

The predetermined range of the thickness W2 of the upper portion 24a is defined in relation to the size of the outer shape of the fine particles 28A. Note that the thickness of the upper portion 24a is equal to the thickness of the lower portion 24b. Hereinafter, the thickness of the upper portion 24a and the thickness of the lower portion 24b are referred to as the “thickness of the inner surface portion 24”. As the size of the outer shape of the fine particles 28A, for example, the maximum value (d50) of the particle size distribution of the fine particles is used. The diameter φ1 corresponding to d50 is defined as the size of the fine particles 28A. The definition of “diameter” of diameter φ1 is the equivalent diameter of a sphere. The diameter φ1 is measured using, for example, a laser diffraction type particle size distribution measuring device. In addition, unlike this embodiment, the diameter φ1 may be an average particle diameter. Since FIG. 9 is a conceptual view, for convenience of explanation, only a single fine particle 28A having a diameter of φ1 is displayed, but in reality, the maximum value is set to a diameter of φ1 on the inner surface portion 24. A group of particles having a predetermined particle size distribution (typically a normal distribution) is dispersed.

The diameter φ1 of the fine particles 28A is defined in a predetermined range, for example, 10 nanometers (nm) or more and less than 100 nanometers, preferably 10 nanometers or more and less than 80 nanometers, more preferably 10 nanometers or more and 40 nanometers or less, and more preferably 10 nanometers or more and 20 nanometers or less.

The outer shape of the fine particles 28A is, for example, spherical. However, the outer shape is not limited to a spherical shape. As the fine particles 28A, for example, copper particles of the “copper nanoparticle SFCP series” manufactured by Fukuda Metal Foil Powder Industry Co., Ltd. (20 Nishinoyama Nakatomi-cho, Yamashina-ku, Kyoto City) can be used. Alternatively, as the fine particles 28A, cuprous copper oxide particles having a primary particle size of about 50 nanometers (nm) as manufactured by Furukawa Chemicals Co., Ltd. (3-7-196 Ono, Nishiyodogawa-ku, Osaka City, Osaka Prefecture) may be used.

The thickness W2 of the inner surface portion 24 is larger than the diameter φ1 of the fine particles 28A, and further larger than the fine particles 28A having the largest particle size in the particle size distribution. As a result, the fine particles 28A are surely covered with the resin 26 and are not exposed on the surface of the inner surface portion 24, and the content of the fine particles 28A on the inner surface portion 24 can be secured within a predetermined range. In this embodiment, the diameter φ1 is 50 nanometers (nm). In the present embodiment, the predetermined range of the content of the fine particles 28A on the inner surface portion 24 is 20% by weight (wt %) or more and 75% by weight or less, preferably 40% by weight (wt %) or more and 75% by weight or less, and even more preferably 60% by weight or more and 75% by weight or less. In this embodiment, the content of the fine particles 28A is 65% by weight.

As described above, the thickness W2 of the inner surface portion 24 is formed to have a thickness within a predetermined range defined relative to the size of the outer shape of the fine particles 28A. If the thickness W2 is too large, the bacterial reduction effect of the fine particles 28A on the side in contact with the main body portion 22 cannot be fully utilized. On the other hand, if the thickness W2 is too small, the fine particles 28A for exhibiting the bacterial reduction effect cannot be sufficiently filled. Therefore, the thickness W2 is defined in a predetermined range relative to the size of the outer shape of the fine particles 28A.

In the present specification, the ratio B (W2/φ1) of the thickness W2 to the diameter φ1 is referred to as “diameter ratio B”. The relationship between the thickness W2 and the diameter φ1 is shown as a numerical range of the diameter ratio B.

The diameter ratio B is defined as a range in which the bacterial reduction effect of the fine particles 28A can be suitably utilized. For example, when the diameter φ1 becomes smaller, the specific surface area of each fine particle 28A becomes large, and since the total surface area of the fine particle group as an aggregate of a large number of fine particles 28A becomes large, the surface area for copper ions to flow out from becomes large. Therefore, the smaller the diameter φ1, the smaller the diameter ratio B may be.

The diameter ratio B is 100 or more and 10000 or less, preferably 100 or more and 5000 or less, more preferably 100 or more and 1000 or less, and more preferably 100 or more and 500 or less. The thickness W2 is defined in the diameter ratio B in the above numerical range with respect to a specific diameter φ1. In the present embodiment, the diameter φ1 of the fine particles is 50 nanometers, the thickness W2 is 20 micrometers, and the diameter ratio B is 400.

FIG. 10 is a conceptual view illustrating in an enlarged manner a portion B2 of the lower portion 24b of the inner surface portion 24 illustrated in FIG. 8. The lower portion 24b is configured by dispersing silver fine particles 28B in the resin. The fine particles 28B are not exposed on the surface of the lower portion 24b and are covered with the resin 26. In the present specification, it is given that “silver” includes silver and silver oxide.

The diameter of the silver fine particles 28B is equal to the diameter φ1 of the copper fine particles 28A. That is, the particle group composed of copper fine particles 28A in the upper portion 24a and the particle group composed of silver fine particles 28B in the lower portion 28b have substantially the same particle size distribution.

The outer shape of the fine particles 28B is spherical. As the fine particles 28B, for example, silver particles of “DOWA AG Nano powders” manufactured by DOWA Electronics Co., Ltd. (4-14-1 Sotokanda, Chiyoda-ku, Tokyo) can be used.

A method of dispersing the copper fine particles 28A and the silver fine particles 28B in the resin, for example, as described above, is carried out by preparing a mixed powder in which a predetermined amount of copper fine particles 28A or silver fine particles 28B are dispersed in the resin powder and then melting and forming the mixed powder through injection molding.

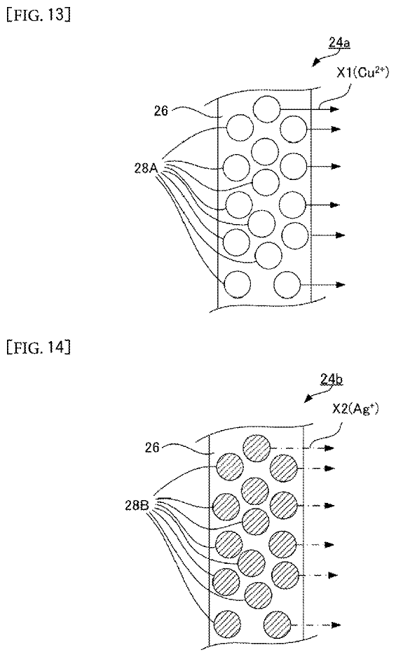

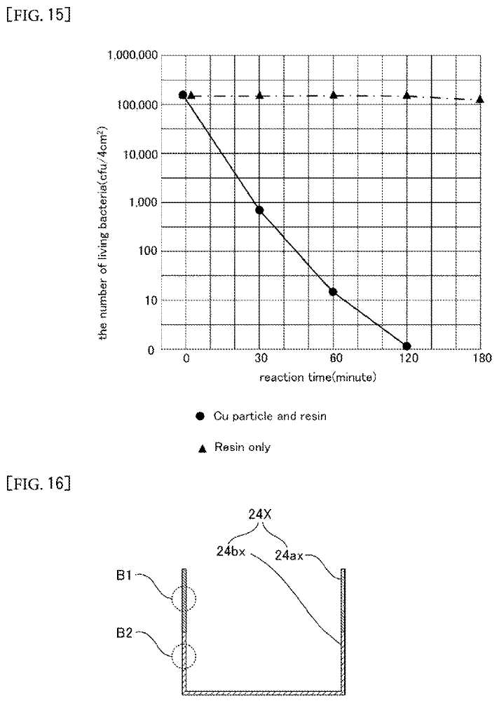

As illustrated in FIG. 11, as the cosmetic product 100 is stored in the container main body 20, copper ions are released from the copper fine particles 28A configuring the upper portion 24a as indicated by arrows X1 in FIGS. 12 and 13, passes through the resin 26, comes into contact with the cosmetic product 100, acts upon the bacteria in the cosmetic product 100 and reduces the bacteria.

The copper fine particles 28A have an effect of reducing bacteria even if they are not in direct contact with the cosmetic product 100. Although the technical field is different from that of the present invention and the configuration is completely different, this is described in JP 4175486, for example. This is also confirmed by the testing results shown in FIG. 15. For example, FIG. 15 shows the testing results of an experiment by Professor Sasai of Kitasato University. As shown in FIG. 15, in the cosmetics container which is a resin containing copper fine particles and the copper fine particles are not exposed, the viable cell count initially present at about 100,000 cfu/4 cm², but after 120 minutes, it has disappeared. Note that “cfu” means “colony forming unit”.

Since it is the resin 26 that comes into contact with the cosmetic product 100 and the fine particles 28A do not come into contact with the cosmetic product 100, they do not react with the components of the cosmetic product 100. That is, by arranging the resin 26 between the copper fine particles 28A and the cosmetic product 100, it is possible to reduce bacteria without denaturing the cosmetic product 100. In addition, it is possible to refrain from adding any preservatives that are usually added to the cosmetic product 100. Alternatively, it is possible to reduce the amount of preservative added.

Further, as illustrated in FIG. 11, as the cosmetic product 100 is stored in the container main body 20, silver ions are released from the silver fine particles 28B configuring the lower portion 24b as indicated by arrows X2 in FIGS. 12 and 14, pass through the resin 26, come into contact with the cosmetic product 100, act upon the bacteria in the cosmetic product 100, and reduce the bacteria.

Similar to the copper fine particles 28A, the silver fine particles 28B have the effect of reducing bacteria even if they are not in direct contact with the cosmetic product 100.

The inventor of the present invention has found that the types of bacteria that can be effectively reduced differ depending on the type of metal. Specifically, the inventor has found that the types of metals that can effectively reduce aerobic bacteria and the types of metals that can effectively reduce anaerobic bacteria are different. In addition, the inventor of the present invention has come up with a technique (“selective dispersion”) in which the type of metal effective for reducing the type of bacteria is determined according to the type of bacteria, and the fine particles of that metal are dispersed in the sterilizing portion. As for copper and silver, copper can effectively reduce aerobic bacteria and silver can effectively reduce anaerobic bacteria.

When the cosmetic product 100 is stored in the cosmetics container 1, the upper portion of the container main body 20 is easily in contact with air, so that it is assumed that aerobic bacteria are mainly a problem. Meanwhile, since the lower portion of the container main body 20 does not easily come into contact with air, it is assumed that anaerobic bacteria are mainly a problem.

Aerobic bacteria are, for example, mold, Pseudomonas aeruginosa, Streptomyces carbophilus, and Bacillus subtilis. Anaerobic bacteria are, for example, Clostridium perfringens and Clostridium botulinum. In the present embodiment, copper fine particles capable of effectively reducing aerobic bacteria are dispersed in the upper portion 24a, and silver fine particles capable of effectively reducing anaerobic bacteria are dispersed in the lower portion 24b. As a result, bacteria that are expected to cause problems can be effectively reduced depending on the vertical position of the cosmetics container 1 in the container main body 20.

Regarding the metal fine particles dispersed in the resin, the specific surface area is important. When ease of actual handling during production is not taken into consideration, it is desirable that the smaller the particle size, the larger the specific surface area. Given that the effect of reducing bacteria when a reference particle size is used as a reference effect, in the case where those having a small particle size are used, the reference effect can be achieved even if the total amount of particles is reduced. The reference particle diameter is given as a particle diameter D1, and a particle diameter smaller than that is defined as a particle diameter D2. It is given that the content of the metal particles having the particle diameter D1 per unit weight of the resin is a content W1 and that it is possible to achieve the reference effect. Given this, it is possible for the reference effect to be achieved by the metal particles having a particle diameter D2 per unit weight of the resin and with a content W2 which is a content smaller than the content W1.

### Second Embodiment

Next, a second embodiment will be described with reference to FIGS. 16 to 18. Descriptions will focus on items differing from the first embodiment; descriptions of items in common will be omitted.

As illustrated in FIG. 16, the inner surface portion 24X is configured of an upper portion 24ax and a lower portion 24bx. Both the upper portion 24ax and the lower portion 24bx are configured by dispersing a plurality of types of metals in a resin. The upper portion 24ax and the lower portion 24bx differ in the content of fine particles of each metal with respect to the content of all metals.

The plurality of types of metals are copper and silver in the present embodiment. While both the upper portion 24ax and the lower portion 24bx contain copper fine particles and silver fine particles, the ratio of the copper fine particle content to the content of all metal fine particles Xcu and the ratio of the of silver fine particle content to the content of all metal fine particles Xag are different. In the present embodiment, as in the first embodiment, the particle size distribution of the copper fine particles and the particle size distribution of the silver fine particles are substantially the same. Therefore, regarding the content, the weight of the fine particles of copper and the weight of the fine particles of silver are used, respectively, per unit weight of the resin. That is, the ratio Xcu of copper fine particles is the ratio Xcu=(weight of Cu)/(weight of Au and Cu), and the ratio Xag of silver particles is the ratio Xag=(weight of Ag)/(weight of Au and Cu). Since the specific gravity of copper is different from that of silver, the content of copper and silver may be specified based on specific gravity, unlike the present embodiment. For example, given that the specific gravity of copper is 8.5 g/cm³ and the specific gravity of silver is 10.5 g/cm³, when 8.5 g of copper and 10.5 g of silver form all metal fine particles, it may be said that the silver and copper content is the same.

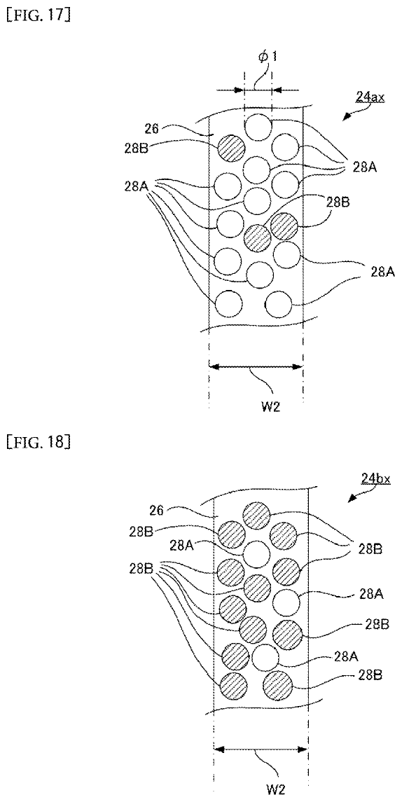

As illustrated in FIG. 17, in the upper portion 24ax, the content of the copper fine particles 28A is higher than the content of the silver fine particles 28B. In addition, as illustrated in FIG. 18, in the lower portion 24bx, the content of the silver fine particles 28B is higher than the content of the copper fine particles 28A. That is, the proportion Xcu in the upper portion 24ax is larger than the proportion Xcu in the lower portion 24bx, and the ratio Xag in the lower portion 24bx is larger than the proportion Xag in the upper portion 24ax. In addition, in FIGS. 17 and 18, the magnitude of the content is expressed by the magnitude of the number of the fine particles 28A and 28B. This also applies to other drawings.

A method for realizing a predetermined ratio of Xcu and Xag, for example, is carried out by preparing a mixed powder in which a predetermined amount of copper fine particles 28A and silver fine particles 28B are dispersed in a resin powder and melting and molding the mixed powder through injection molding.

The type of bacteria that is expected to cause a problem at a specific position on the inner surface portion 24X is not limited to one type. The inventor of the present invention discovered that at a specific position on the inner surface portion 24X, there exist a plurality of types of bacteria that are assumed to be problematic, and further, depending on the specific position, the specific type of bacteria that is relatively abundant varies.

In addition, the inventor arrived at a technique of mixing metal fine particles to effectively reduce each type of bacteria, which are expected to cause problems at a specific position and dispersing them throughout the sterilizing portion (“load distribution”).

In the present embodiment, while aerobic bacteria and anaerobic bacteria can be a problem in both the upper portion 24ax and the lower portion 24bx, it is assumed that a relatively large number of aerobic bacteria can be present in the upper portion 24ax and it is assumed that a relatively large number of anaerobic bacteria may be present in the lower portion 24bx. Since the content of the copper fine particles in the upper portion 24ax is larger than the content of the silver fine particles and the content of the silver fine particles in the lower portion 24bx is larger than the content of the copper fine particles, it is possible to effectively reduce each of the bacteria depending on the plurality of types of bacteria expected to be problematic at a specific location on the inner surface portion 24X.

### Third Embodiment

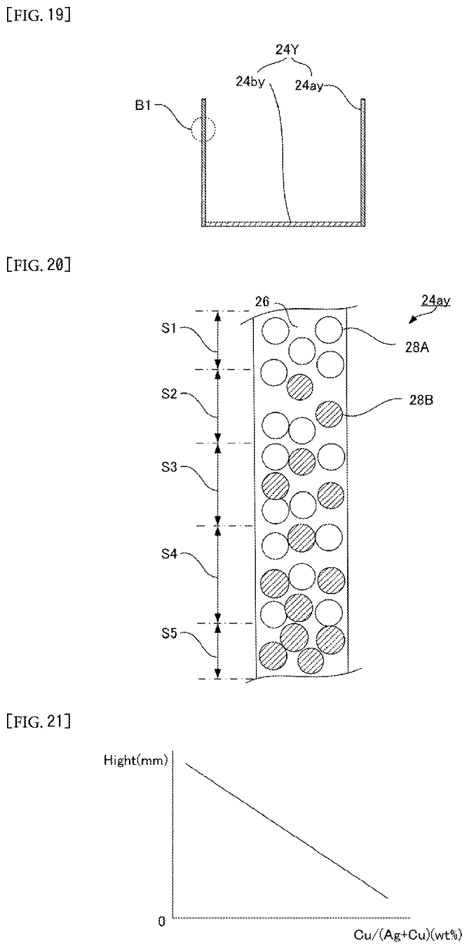

Next, a third embodiment will be described with reference to FIGS. 19 to 21. Descriptions will focus on items differing from the first embodiment and second embodiment; descriptions of items in common will be omitted.

As illustrated in FIG. 19, in an inner surface portion 24Y, an inner peripheral portion 24ay contains metal fine particles having a property of effectively reducing aerobic bacteria and metal fine particles having a property of effectively reducing anaerobic bacteria. The inner peripheral portion 24ay contains a larger amount of metal fine particles having a property of effectively reducing aerobic bacteria toward the upper side and is configured so that the content of metal fine particles having a property of effectively reducing aerobic bacteria decreases from the upper side toward the lower side. Meanwhile, the inner peripheral portion 24ay is configured so that the content of metal fine particles having a property of effectively reducing anaerobic bacteria increases from the upper side toward the lower side.

As illustrated in FIG. 20, when the inner peripheral portion 24ay is divided into the regions S1 to S5 from the upper side toward the lower side, from region S1 toward region S5, the content of the copper fine particles 28A decreases and the content of silver fine particles 28B increases. Conversely, from region S5 toward region S1, the content of the copper fine particles 28A increases and the content of the silver fine particles 28B decreases. In order to configure the distribution of the metal particles in the inner peripheral portion 24ay in this way, for example, specific gravity is utilized. For example, with injection molding, the fact that the specific gravity of copper is smaller than the specific gravity of silver is utilized.

More specifically, as illustrated in FIG. 21, when the distance from the bottom portion 24by is defined as the height, the taller the height, the greater the proportional content of the copper fine particles of the total metal particles. Conversely, the shorter the height, the greater the proportional content of silver fine particles of the total metal particles. The bottom portion 24by is configured by dispersing only silver fine particles 28B in the resin. This is because it is assumed that anaerobic bacteria will be the main problem at the bottom portion 24by.

When the cosmetic product 100 is stored in the container main body 1, of the total bacteria assumed to become a problem, it is thought that the proportion of aerobic bacteria decreases, and the proportion of anaerobic bacteria increases, toward the lower portion of the container main body 1. In this regard, according to the present embodiment, it is possible to effectively reduce bacteria at each position according to the ratio of aerobic bacteria and anaerobic bacteria which are assumed to become a problem.

### Fourth Embodiment

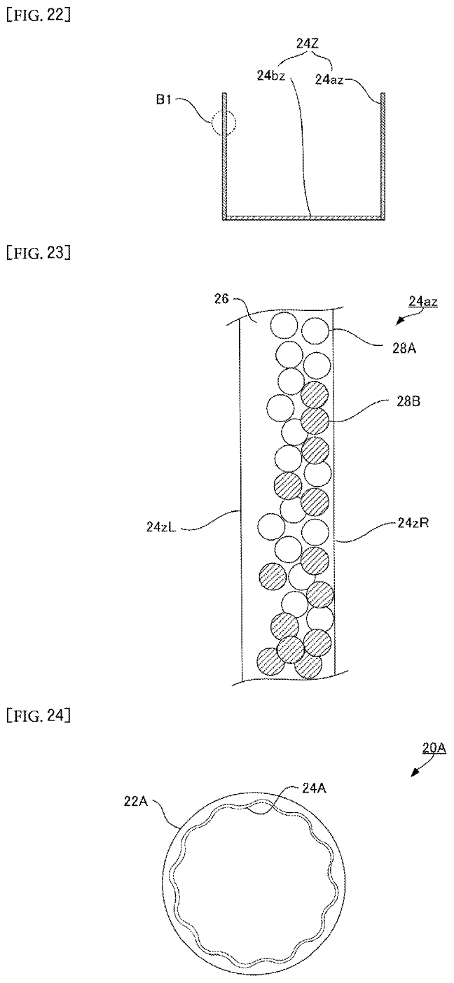

Next, a fourth embodiment will be described with reference to FIGS. 22 and 23. Descriptions will focus on items differing from the first embodiment through third embodiment; descriptions of items in common will be omitted.

An inner peripheral portion 24az configuring an inner surface portion 24Z is configured by dispersing metal fine particles 28A and 28B in the resin 26. The inner peripheral portion 24az will be described with reference to FIG. 23. One surface 24zR of the inner peripheral portion 24az comes into contact with the cosmetic product. The metal fine particles 28A and 28B are contained in a larger amount at the position on the surface 24zR side than at the position on the opposite surface 24zL side.

The closer the metal fine particles are to the surface in contact with cosmetic product, the greater the effect of reducing bacteria. In this respect, according to the configuration of the present embodiment, since the metal fine particles are contained in a larger amount at the position on the side of the surface in contact with the cosmetic product than on a position opposite to the surface in contact with the cosmetic product, it is possible to effectively reduce the bacteria.

A technique for distributing more metal fine particles on one surface of the inner surface portion 24Z, for example, is carried out by preparing a mixed powder in which a predetermined amount of copper fine particles 28A and silver fine particles 28B are dispersed in a resin powder and melting and molding the mixed powder through injection molding in an electric field or a magnetic field. Alternatively, it may be carried out by appropriately adjusting the composition of the mixed powder. The composition of the mixed powder includes, but is not limited to, a composition of additives and lubricants.

Unlike the present embodiment, the inner surface portion 24Z may be configured to contain fine particles of a single kind of metal. For example, the inner surface portion 24Z may be configured to contain only one of either the copper fine particles 28A or the silver fine particles 28B.

### Fifth Embodiment

Next, a fifth embodiment will be described with reference to FIG. 24. Descriptions will focus on items differing from the first embodiment through fourth embodiment; descriptions of items in common will be omitted.

As illustrated in FIG. 24, a side surface of an inner surface portion 24A of a container main body 20A of the fifth embodiment is formed as an uneven surface having a plurality of convex portions and concave portions. As a result, the area of the inner surface portion 24A in contact with the cosmetic product 100 can be increased in comparison with the case where the inner surface portion 24A is a curved surface having no unevenness. As for the dispersion mode of the metal fine particles on the inner surface portion 24A, any mode of the first embodiment through the fourth embodiment is applicable.

The thickness W2 of the inner surface portion 24A is the same as that of the first embodiment. That is, the inner surface of the main body portion 22A is also formed as an uneven surface, and the inner surface portion 24A is integrally formed with the inner surface of the main body portion 22A.

In addition, unlike the present embodiment, both the side surface and a bottom surface of the inner surface portion 24A may be formed as an uneven surface having a plurality of convex portions and concave portions, or only the bottom surface may be formed as such an uneven surface.

### Sixth Embodiment

Next, a sixth embodiment will be described with reference to FIG. 25. Descriptions will focus on items differing from the first embodiment through fourth embodiment; descriptions of items in common will be omitted.

An inner surface portion 24B of a container main body 20B of a cosmetics container of the sixth embodiment is arranged only on a side surface. That is, the main body portion 22 has a side surface and a bottom surface, and the sterilizing layer is arranged only on the side surface. In addition, if the container main body has an elongated shape with a small bottom surface unlike the container main body 20B of FIG. 25, it is possible to effectively reduce bacteria by arranging the inner surface portion 24B only on the side surface. As for the dispersion mode of the metal fine particles on the inner surface portion 24B, any mode of the first embodiment through the fourth embodiment is applicable.

### Seventh Embodiment

Next, a seventh embodiment will be described with reference to FIG. 26. Descriptions will focus on items differing from the first embodiment through fourth embodiment; descriptions of items in common will be omitted.

An inner surface portion 24C of a container main body 20C of a cosmetics container of the seventh embodiment is arranged only on a bottom surface. That is, the main body portion 22 has a side surface and a bottom surface, and the sterilizing layer is arranged only on the bottom surface. In addition, unlike the container main body 20C of FIG. 26, if the container main body has a smaller depth and a flatter shape, it is possible to effectively reduce bacteria by arranging the inner surface portion 24C only on the bottom surface. The inner surface portion 24C is configured by dispersing metal fine particles suitable for reducing anaerobic bacteria in the resin. The metal fine particles are, for example, silver fine particles. As in the fourth embodiment, it is desirable that the silver fine particles are dispersed in a larger amount on the side closer to the surface in contact with the cosmetic product 100.

### Eighth Embodiment

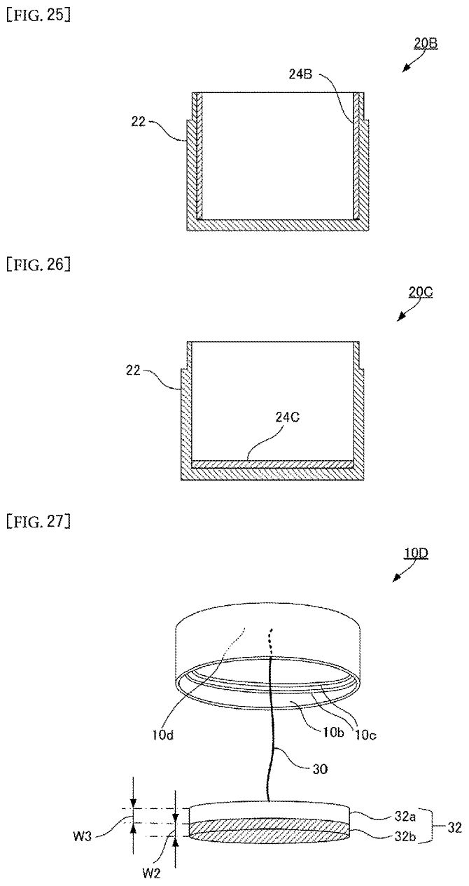

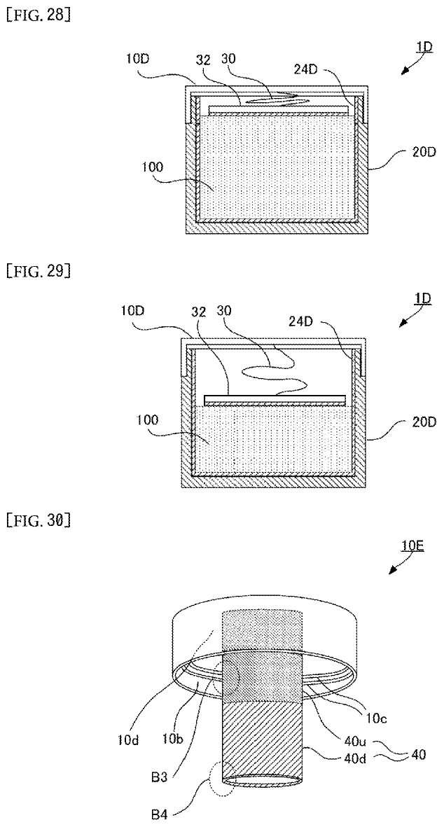

Next, an eighth embodiment will be described with reference to FIGS. 27 through 29. Descriptions will focus on items differing from the first embodiment through fourth embodiment; descriptions of items in common will be omitted.

As illustrated in FIG. 27, an inner lid 32 is connected to a lid body 10D via a string-shaped member 30. The string-shaped member 30 is an example of a connecting member. The string-shaped member 30 is, for example, a string made of polyester. The connecting member is not limited to the string-shaped member, and for example, a spring may be adopted.

The inner lid 32 is configured by integrally forming an inner lid main body portion 32a configuring the upper portion and an inner lid lower portion 32b configuring the lower portion. The thickness W3 of the inner lid main body portion 32a is larger than the thickness W2 of the inner lid lower portion 32b. The inner lid main body portion 32a functions as a structural member of the inner lid 32, and also functions as a member having a weight for pressing the inner lid 32 against the cosmetic product 100. The thickness W3 of the inner lid main body portion 32a is, for example, 5 mm. The thickness W2 of the inner lid lower portion 32b is, for example, 20 micrometers.

The inner lid lower portion 32b is configured as a sterilizing portion that reduces bacteria by the action of the copper fine particles 28A by dispersing the copper fine particles 28A (see FIG. 9) in the resin and covering the copper fine particles 28A with the resin 26 (see FIG. 9) without being exposed on the surface. That is, in addition to the inner surface portion 24 in the first embodiment, the inner lid lower portion 32b exists as a second sterilizing portion.

The thickness W2 of the inner lid lower portion 32b is defined within a predetermined range defined in relation to the outer size of the copper fine particles 28A. The method for defining the predetermined range is the same as the method for defining the thickness W2 of the inner surface portion 24 of the first embodiment.

The length of the string-shaped member 30 is defined as the length at which the inner lid 32 is able to come into contact with the bottom surface of the container main body 20D.

FIG. 28 illustrates a state in which an amount of cosmetic product 100 close to the storage limit of a container main body 20D is held. When the lid body 10D engages with the container main body 20D, the inner lid 32 comes into contact with the upper surface of the cosmetic product 100. At this time, the inner lid lower portion 32b is pressed against the upper surface of the cosmetic product 100 by the weight of the inner lid main body portion 32a. As a result, the inner lid lower portion 32b comes into close contact with the upper surface of the cosmetic product 100, and it is possible to effectively reduce bacteria. there is a possibility that aerobic bacteria may grow on the upper surface of the cosmetic product 100. Therefore, the inner lid lower portion 32b is configured by dispersing metal fine particles suitable for reducing aerobic bacteria in the resin. For the inner surface 24D, the configuration of any of the first through fourth embodiments is applicable.

FIG. 29 illustrates a state in which the cosmetic product 100 stored in the container main body 20D is consumed and reduced. Since the inner lid 32 is connected to the lid body 10D via the string-shaped member 30, even if the amount of cosmetic product 100 in the container main body 20D is reduced, when the lid body 10D engages with the container main body 20D, the inner lid 32 comes into contact with the upper surface of the cosmetic product 100. As described above, regardless of the amount of cosmetic product 100 remaining, it is possible for the inner lid lower portion 32b to come into close contact with the upper surface of the cosmetic product 100, and effectively reduce bacteria.

Unlike the present embodiment, when a compression coil spring is used as the connecting member, since it is possible for the inner lid 32 to be actively pressed against the upper surface of the cosmetic product 100 through the restorative force of the spring, it is possible to increase the degree of close contact. Further, unlike the present embodiment, the lower surface (the surface in contact with the cosmetic product 100) of the inner lid lower portion 32b may be configured as an uneven surface having a plurality of concave portions and convex portions, so as to configure having a larger surface area in contact with the cosmetic product 100.

Further, unlike the present embodiment, the container main body 20D may be configured without a sterilizing portion and configured with only the inner lid lower portion 32b of the inner lid 32 configured as a sterilizing portion. Alternatively, unlike the present embodiment, the inner lid lower portion 32b may be configured to contain a plurality of types of metal fine particles and configured having a larger content of the metal fine particles that suitably reduce aerobic bacteria than the content of the metal fine particles that suitably reduce anaerobic bacteria.

### Ninth Embodiment

Next, a ninth embodiment will be described with reference to FIGS. 30 to 34. Descriptions will focus on items differing from the first embodiment through fourth embodiment; descriptions of items in common will be omitted.

A tubular member 40 is connected to a lid body 10E. The tubular member 40 is a member having a hollow structure. The tubular member 40 is configured of an upper portion 40u which is the portion above and a lower portion 40d which is the portion below.

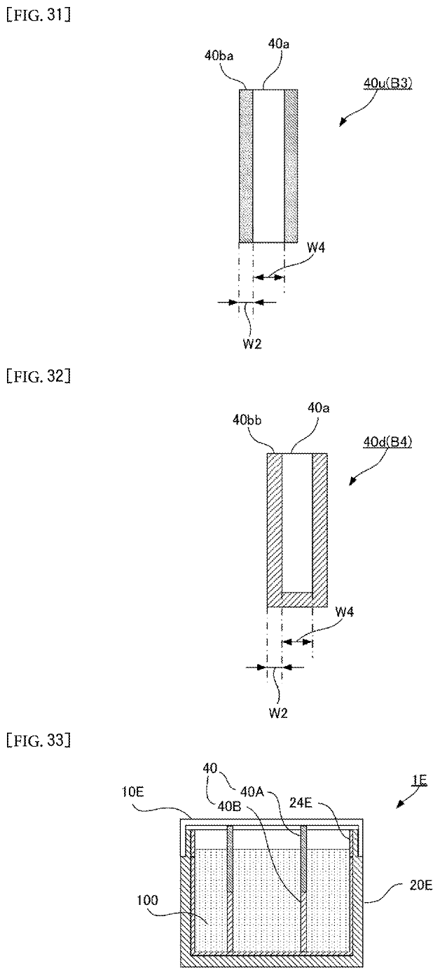

FIG. 31 is a schematic cross-sectional view of a peripheral wall portion B3 of the upper portion 40u of the tubular member 40 illustrated in FIG. 30. The peripheral wall of the upper portion 40u is comprised of a central portion 40a and a surface layer portion 40ba. The central portion 40a functions as a structural member of the tubular member 40, with the thickness W4 thereof being a thickness having the strength necessary for maintaining the shape of the tubular member 40, and being, for example, 2 millimeters (mm). The surface layer portion 40ba is configured as a sterilizing portion that reduces bacteria through the action of the copper fine particles 28A by dispersing the copper fine particles 28A in the resin and covering the copper fine particles 28A with a resin 26 so as not to be exposed on the surface.

The thickness W2 of the surface layer portion 40ba is defined within a predetermined range defined in relation to the size of the copper fine particles 28A. The method for defining the predetermined range is the same as the method for defining the thickness W2 of the inner surface portion 24 of the first embodiment.

FIG. 32 is a schematic cross-sectional view of the peripheral wall portion B4 of the lower portion 40d of the tubular member 40 illustrated in FIG. 30. The lower portion 40d has the same configuration as the upper portion 40u except for the following points. That is, a surface layer portion 40bb of the lower portion 40d is formed so as to cover the side surface and the bottom surface of the central portion 40a. Further, the surface layer portion 40bb is configured as a sterilizing portion that reduces bacteria through the action of the silver fine particles 28B by dispersing the silver fine particles 28B in the resin.

As described above, it is configured that the upper portion 40u is suitable for reducing aerobic bacteria, and the lower portion 40d is suitable for reducing anaerobic bacteria. That is, in addition to the inner surface portion 24 in the first embodiment, the tubular member 40 exists as a second sterilizing portion.

As illustrated in FIG. 33, the length of the tubular member 40 is defined as a length allowing contact with the bottom surface of the container main body 20E. FIG. 33 shows a state in which an amount of cosmetic product 100 close to the storage limit of the container main body 20E is stored. When the lid body 10E engages with the container main body 20E, the tubular member 40 passes through the cosmetic product 100 and comes into contact with the bottom surface of the container main body 20E. At this time, both the inner surface and the outer surface of the tubular member 40 come into contact with the cosmetic product 100. As a result, it is possible to effectively reduce bacteria.

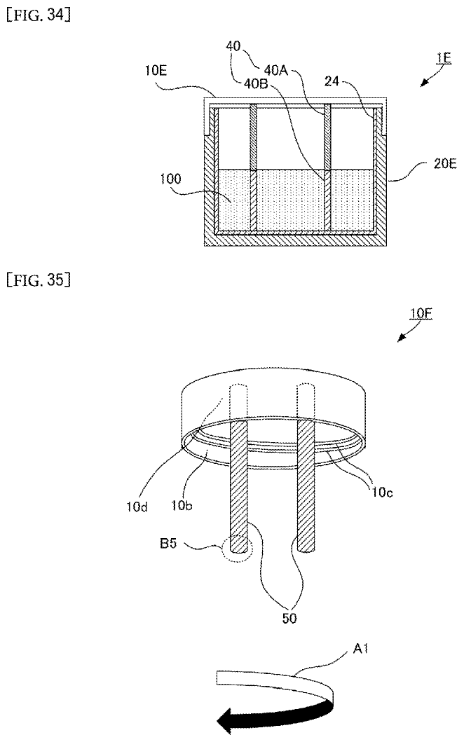

FIG. 34 illustrates a state in which the cosmetic product 100 stored in the container main body 20E has been consumed and become reduced. Since the tubular member 40 is in contact with the bottom surface of the container main body 20E, even if the cosmetic product 100 in the container main body 20E becomes reduced, when the lid body 10E engages with the container main body 20E, the tubular member 40 comes into contact with the cosmetic product 100. In this way, regardless of amount of cosmetic product 100 remaining, it is possible for the tubular member 40 to come into contact with the cosmetic product 100 and effectively reduce bacteria.

In addition, unlike this embodiment, the configuration of the surface layer portion 40ba and/or 40bb of the tubular member 40 may use any of the modes of the second embodiment to the fourth embodiment.

### Tenth Embodiment

Next, a tenth embodiment will be described with reference to FIGS. 35 to 38. Descriptions will focus on items differing from the first embodiment through fourth embodiment; descriptions of items in common will be omitted.

As illustrated in FIG. 35, two rod-shaped members 50 are connected to a ceiling surface 10d of a lid body 10F. Note that the number of rod-shaped members 50 may be one or more.

FIG. 36 is a schematic cross-sectional view of a portion B5 of a rod-shaped member 50 illustrated in FIG. 35. The rod-shaped member 50 is configured of a central portion 50a and a surface layer portion 50b. The surface layer portion 50b of the rod-shaped member 50 is configured as a sterilizing portion that reduces bacteria by the action of the metal fine particles suitable for reducing bacteria by dispersing the metal fine particles in the resin 26 and covering the metal fine particles so as not to be exposed on the surface. That is, in addition to the inner surface portion 24 according to the first embodiment, the rod-shaped member 50 exists as a second sterilizing portion.

The thickness W2 of the surface layer portion 50b is defined within a predetermined range defined in relation to the size of the outer shape of the copper fine particles 28. The method for defining the predetermined range is the same as the method for defining the thickness W2 of the inner surface portion 24 of the first embodiment. The thickness (diameter) W5 of the central portion 50a is configured to be larger than the thickness W1 of the first embodiment securing sufficient strength to withstand drag force when the cosmetic product 100 is agitated by the rod-shaped member 50. The diameter W5 is, for example, 5 millimeters (mm).

The length of the rod-shaped member 50 is defined as a length that while not coming into contact with the bottom surface of a container main body 20F when the lid body 10F engages with the container main body 20F, has the lowermost portion thereof located near the bottom surface.

As illustrated in FIGS. 35 and 37, the rod-shaped members 50 are connected at positions deviating from the center of the lid body 10F. As a result, when the lid body 10F is rotated for attachment/detachment to/from the container main body 20F, they rotate in a circular motion as indicated by arrow A1 in FIGS. 35 and 37. This configuration allows the cosmetic product 100 stored in the container main body 20F to be agitated.

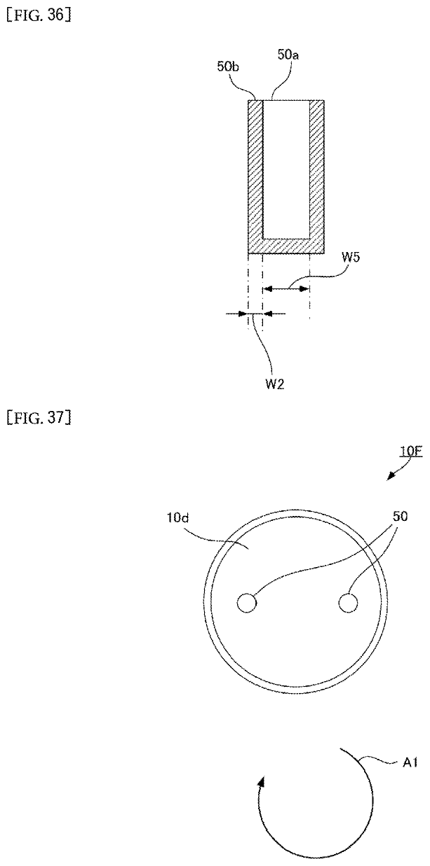

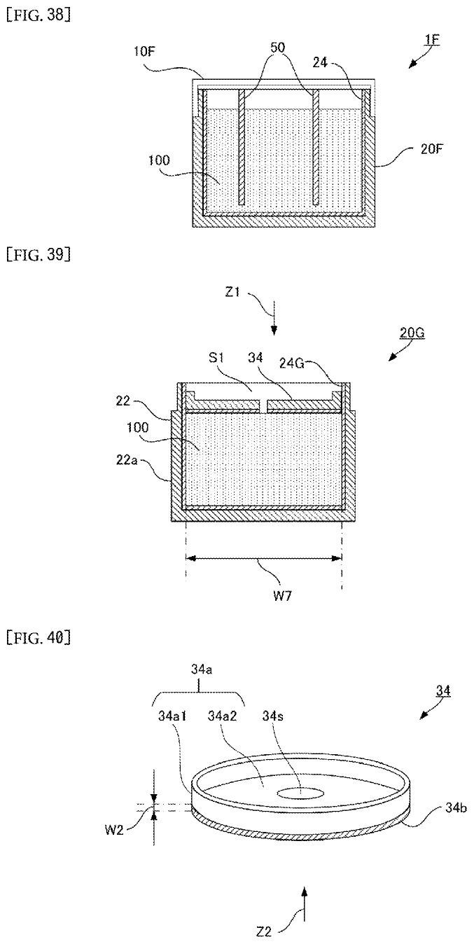

FIG. 38 illustrates a state in which an amount of cosmetic product 100 close to the storage limit of the container main body 20F is held. As the lid body 10F engages with a container main body 20F, the rod-shaped members 50 pass through the cosmetic product 100, and the lowermost portions thereof are located near the bottom surface of the container main body 20F. At this time, the surface layer portion 50b of each rod-shaped member 50 comes into contact with the cosmetic product 100. This makes it possible to effectively reduce bacteria.

Further, as the lid body 10F is removed from the container main body 20F from the state of FIG. 38, the rod-shaped members 50 also rotate with the rotation of the lid body 10F to stir the cosmetic product 100. As a result, the rod-shaped members 50 are able to come into contact with many portions of the cosmetic product 100 and more effectively reduce bacteria.

The configuration of the surface layer portion 50b of each rod-shaped member 50 utilizes any one of the first embodiment through the fourth embodiment.

### Eleventh Embodiment

Next, an eleventh embodiment will be described with reference to FIGS. 39 to 42. Descriptions will focus on items differing from the first embodiment through fourth embodiment; descriptions of items in common will be omitted.

As illustrated in FIG. 39, an inner lid 34 is arranged on a container main body 20G. The inner lid 34 is an example of a main body side inner lid.

As illustrated in FIG. 40, the inner lid 34 is configured by integrally forming an inner lid main body portion 34a configuring an upper portion and an inner lid lower portion 34b configuring a lower portion. The inner lid main body portion 34a is an example of a main body side inner lid main body portion, and the inner lid lower portion 34b is an example of a main body side inner lid lower portion.

The inner lid main body portion 34a is configured of a side wall portion 34a1 and a convex bottom surface portion 34a2. In the inner lid 34, is formed a through hole 34s that penetrates in the vertical direction.

The inner lid lower portion 34b is configured as a sterilizing portion. The inner lid lower portion 34b is configured by dispersing in a resin metal fine particles suitable for reducing aerobic bacteria, for example, copper fine particles 28A.

The thickness W2 of the inner lid lower portion 34b is defined within a predetermined range defined in relation to the size of the copper fine particles 28A. The method for defining the predetermined range is the same as the method for defining the thickness W2 of the inner surface portion 24 of the first embodiment.

The outer diameter W8 of the inner lid 34 (see FIG. 41) is equal to the inner diameter W7 of the container main body 20G or is larger than the inner diameter W7 by a predetermined minimum ratio. The minimum ratio is, for example, from 0.5% to 1.0%. As a result, substantially no gap or only a slight gap is configured between the outer periphery of the inner lid 34 and the inner surface portion 24 of the container main body 20G. The inner lid 34 is arranged so as to be slidable in the vertical direction with respect to the container main body 20G.

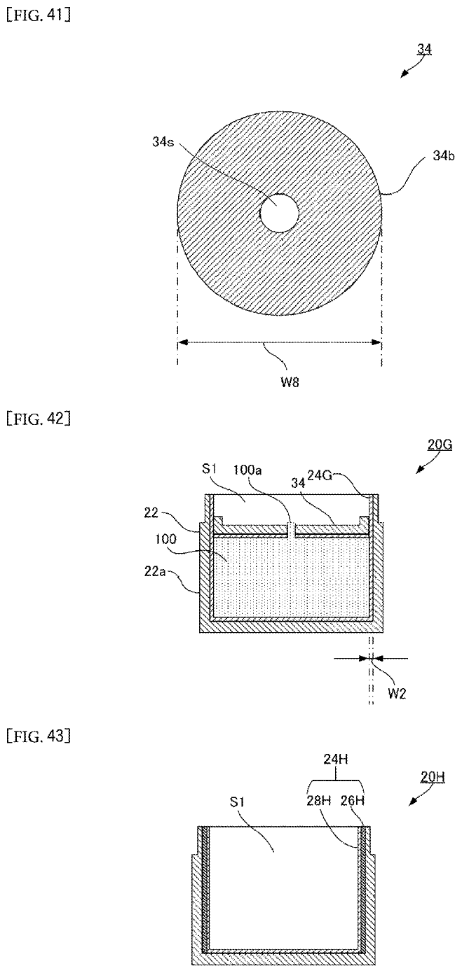

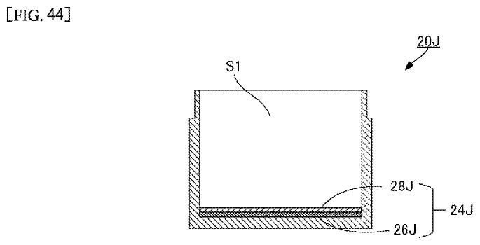

In the state illustrated in FIG. 39, as the inner lid 34 is pressed from above (from the direction of arrow Z1), the inner lid 34 slides and moves downward, and as illustrated in FIG. 42, the cosmetic product 100a is pushed out from the through hole 34s. Since until just before being pushed out from the through hole 34s, the cosmetic product 100a was located below the inner lid 34, it is unlikely to come into contact with external bacteria, and even if bacteria were to have developed, they are reduced by the sterilizing effect of the inner lid lower portion 34b.

In addition, unlike this embodiment, the inner lid lower portion 34b may be configured to contain both copper fine particles 28A and silver fine particles 28B. However, the content of the copper fine particles 28A is made greater than the content of the silver fine particles 28B. Through this, it is possible for the inner lid lower portion 34b to effectively reduce aerobic bacteria while also reducing anaerobic bacteria.

### First Reference Embodiment

Next, a first reference embodiment will be described with reference to FIG. 43. Descriptions will focus on items differing from the first embodiment; descriptions of items in common will be omitted.

An inner surface portion 24H of a container main body 20H of a cosmetics container of the first reference embodiment is formed by covering a thin copper plate member 26H with a resin layer 28H formed of resin. Further, the thin plate member 26H is arranged only on the side surface.

When the cosmetic product 100 is held in the internal space S1, bacteria in the cosmetic product can be reduced by the action of the copper of the thin plate member 26H.

### Second Reference Embodiment

Next, a second reference embodiment will be described with reference to FIG. 44. Descriptions will focus on items differing from the first embodiment; descriptions of items in common will be omitted.

An inner surface portion 24J of a container main body 20J of a cosmetics container of the second reference embodiment is formed by covering a thin copper plate member 26J with a resin layer 28J formed of resin. Further, the thin plate member 26J is arranged only on the bottom surface.

When a cosmetic product is held in the internal space S1, bacteria in the cosmetic product can be reduced through the action of the copper of the thin plate member 26J.

The cosmetics container of the present invention is not limited to the above embodiments, and various modifications can be made without departing from the gist of the present invention. In addition, each of the above embodiments can be appropriately combined as long as there is no technical contradiction.

### REFERENCE SIGNS LIST

1, 1D, 1E, 1F Cosmetics container
10, 10D, 10E, 10F Lid body
20, 20A, 20B, 20C, 20D, 20E, 20F, 20G, 20H, 20J Container main body
22 Main body portion
24, 24X, 24Y, 24Z Inner surface portion
26 Resin
28A Copper fine particles
28B Silver fine particles
32, 34 Inner lid
40 Tubular member
50 Rod-shaped member
100 Cosmetic product

1. A cosmetics container, comprising a container main body for holding a cosmetic product, and a lid body which detachably engages with the container main body, wherein
the container main body is configured of a main body portion configuring the basic structure of the container main body and an inner surface portion configuring an inner surface of the container main body;
the inner surface portion is configured as a sterilizing portion in which metal fine particles having an effect of reducing bacteria are dispersed in a resin and bacteria are reduced by the action of the fine particles;
the fine particles are configured so as not to be exposed on the surface of the inner surface portion; and
the type of the metal configuring the sterilizing portion is defined in accordance with the type of bacteria assumed to become a problem at each position on the inner surface portion.

2. The cosmetics container according to claim 1, wherein
the inner surface portion
includes a plurality of types of the metal fine particles; and
at the each position on the inner surface portion, the content of each of the metal fine particles is defined relative to the total content of the metal fine particles in accordance with the type of bacteria assumed to become a problem.

3. The cosmetics container according to claim 2, wherein
each position on the inner surface portion includes an upper portion located relatively upper and a lower portion located relatively lower;
the type of bacteria expected to be relatively more likely to become a problem in the upper portion is aerobic bacteria, and the type of bacteria expected to be relatively more likely to become a problem in the lower portion is anaerobic bacteria;
the upper portion is configured so that the content of the metal fine particles having a property of effectively reducing the aerobic bacteria is increased; and
the lower portion is configured so that the content of the metal fine particles having a property of effectively reducing the anaerobic bacteria is increased.

4. The cosmetics container according to claim 2, wherein
the inner surface portion is configured so that, from the upper side toward the lower side, the content of the metal fine particles having a property of effectively reducing the aerobic bacteria decreases and the content of the metal fine particles having a property of effectively reducing the anaerobic bacteria increases.

5. The cosmetics container according to claim 1, wherein
in the inner surface portion, the metal fine particles are configured to increase in content as they approach a position close to a surface where the cosmetic product comes into contact with the inner surface portion.

6. The cosmetics container according to claim 1, wherein
a thickness of the inner surface portion is formed to be a thickness within a predetermined range defined in relation to the size of the outer shape of the fine particles.

7. The cosmetics container according to claim 1, wherein
the inner surface portion has a side surface portion and a bottom surface portion and the side surface portion and/or the bottom surface portion is formed as an uneven surface having a plurality of convex portions and concave portions.

8. The cosmetics container according to claim 1, wherein
the inner surface portion has a side surface portion and a bottom surface portion, and only one of the side surface portion or the bottom surface portion is configured as a sterilizing portion.

9. A cosmetics container, comprising a container main body for holding a cosmetic product and a lid body which detachably engages with the container main body, wherein
an inner lid is connected to the lid body via a connecting member;
the inner lid is configured of an inner lid main body portion configuring an upper portion, and an inner lid lower portion configuring a lower portion;
the inner lid lower portion is configured as a sterilizing portion in which metal fine particles having an effect of reducing bacteria are dispersed in a resin and bacteria are reduced by the action of the fine particles;
the inner lid lower portion is configured so that the fine particles are not exposed on the surface of the inner lid lower portion;
a length of the connecting member is defined as an adjustable length which allows the inner lid to come into contact with a bottom surface of the container main body;
by adjusting the length of the connecting member, the inner lid lower portion is configured to come into contact with the cosmetic product regardless of the amount of the cosmetic product held in the container main body; and
the inner lid lower portion is configured to have, among the plurality of types of the metal fine particles, a large content of the metal fine particles having a property of being able to effectively reduce aerobic bacteria.

10. A cosmetics container, comprising a container main body for holding a cosmetic product and a lid body which detachably engages with the container main body, wherein
to the lid body, a tubular member is connected;
a surface layer portion of the tubular member is configured as a sterilizing portion in which metal fine particles having an effect of reducing bacteria are dispersed in a resin and bacteria are reduced by the action of the fine particles;
a surface layer portion of the tubular member is configured so that the fine particles are not exposed on the surface of the surface layer portion;
the length of the tubular member is defined as a length allowing contact with a bottom surface of the container main body;
regardless of the amount of the cosmetic product held in the container main body, the surface layer portion of an inner surface and an outer surface of the tubular member are configured to be in contact with the cosmetic product; and
the type of the metal configuring the sterilizing portion is defined according to the type of bacteria assumed to become a problem at each position on the surface layer portion.

11. A cosmetics container, comprising a container main body for holding a cosmetic product and a lid body which detachably engages with the container main body, wherein
to the lid body, a least one rod-shaped member is connected;
a surface layer portion of the rod-shaped member is configured as a sterilizing portion in which metal fine particles having an effect of reducing bacteria are dispersed in a resin and bacteria are reduced by the action of the fine particles;
the surface layer portion is configured so that the fine particles are not exposed on the surface of the surface layer portion;
the rod-shaped member is connected at a position deviating from the center of the lid body, and as the lid body is rotated for attachment/detachment to/from the container main body, is configured to rotate and stir the cosmetic product held in the container main body; and
the type of the metal configuring the sterilizing portion is defined according to the type of bacteria assumed to become a problem at each position of the surface layer portion.

12. A cosmetics container, comprising a container main body for holding a cosmetic product and a lid body which detachably engages with the container main body, wherein
on the container main body, a main body side inner lid is arranged;
the main body side inner lid
has a main body side inner lid main body portion configuring an upper portion and a main body side inner lid lower portion configuring a lower portion, and
is formed with a through hole passing through the main body side inner lid in the vertical direction;
the main body side inner lid lower portion is configured as a sterilizing portion in which metal fine particles having an effect of reducing bacteria are dispersed in a resin and bacteria are reduced by the action of the fine particles;
the main body side inner lid lower portion is configured so that the fine particles are not exposed on the surface of the main body side inner lid lower portion;
the main body side inner lid lower portion is configured to come into contact with the cosmetic product, and by moving the main body side inner lid so as to push on the cosmetic product, the cosmetic product is able to be pushed out from the through hole; and
the main body side inner lid lower portion is configured so that of the plurality of types of the metal fine particle, the content of the metal fine particles having a property of effectively reducing aerobic bacteria is large.

13. An inner lid connected to a lid body which detachably engages with a container main body for holding a cosmetic product, wherein
the inner lid is
connected to the lid body via a connecting member, and
configured of an inner lid main body portion configuring an upper portion and an inner lid lower portion configuring a lower portion;
the inner lid lower portion is configured as a sterilizing portion in which metal fine particles having an effect of reducing bacteria are dispersed in the resin and bacteria are reduced by the action of the fine particles;
the inner lid lower portion is configured so that the fine particles are not exposed on the surface of the inner lid lower portion;
the length of the connecting member is defined as an adjustable length that allows the inner lid to come into contact with a bottom surface of the container main body;
by adjusting the length of the connecting member, the inner lid lower portion is configured to come into contact with the cosmetic product regardless of the amount of the cosmetic product held in the container main body; and
the inner lid lower portion is configured so that of the plurality of types of the metal fine particles, the content of the metal fine particles having a property of effectively reducing aerobic bacteria is large.

14. A tubular member connected to a lid body that detachably engages with a container main body for holding cosmetic product, wherein
a surface layer portion of the tubular member is configured as a sterilizing portion in which metal fine particles having an effect of reducing bacteria are dispersed in a resin and bacteria are reduced by the action of the fine particles;
the surface layer portion is configured so that the fine particles are not exposed on the surface of the surface layer portion;
a length of the tubular member is defined as a length that allows contact with a bottom surface of the container main body;
the surface layer portions of an inner surface and an outer surface of the tubular member are configured to be in contact with the cosmetic product regardless of the amount of the cosmetic product held in the container main body; and
the metal configuring the sterilizing portion is defined according to the type of bacteria assumed to become a problem at each position on the surface layer portion.

15. A rod-shaped member connected to a lid body that detachably engages with a container main body for holding cosmetic product, wherein
a surface layer portion of the rod-shaped member is configured as a sterilizing portion in which metal fine particles having an effect of reducing bacteria are dispersed in a resin and bacteria are reduced by the action of the fine particles;
the surface layer portion is configured so that the fine particles are not exposed on the surface of the surface layer portion;
the rod-shaped member is connected at a position deviating from the center of the lid body, and as the lid body is rotated for attachment/detachment to/from the container main body, is configured to rotate and stir the cosmetic product held in the container main body; and
the metal configuring the sterilizing portion is defined according to the type of bacteria assumed to become a problem at each position on the surface layer portion.

16. A main body side inner lid that is arranged on a container for holding cosmetic product, wherein
the main body side inner lid is configured by
integrally forming a main body side inner lid main body portion configuring an upper portion and a main body side inner lid lower portion configuring a lower portion, and
a through hole is formed so as to pass through in the vertical direction the main body side inner lid;
the main body side inner lid lower portion is configured as a sterilizing portion in which metal fine particles having an effect of reducing bacteria are dispersed in a resin and bacteria are reduced by the action of the fine particles;
the main body side inner lid lower portion is configured so that the fine particles are not exposed on the surface of the main body side inner lid lower portion;
the main body side inner lid lower portion is configured to come into contact with the cosmetic product, and by moving the main body side inner lid so as to push on the cosmetic product, the cosmetic product is able to be pushed out from the through hole; and
the inner lid lower portion is configured so that of the plurality of types of the metal fine particles, the content of the metal fine particles having a property of effectively reducing aerobic bacteria is large.
